(19) United States
(12) Patent Application Publication
(10) Pub. No.: US 2026/0179324 A1
(43) Pub. Date: Jun. 25, 2026

(54) METHOD AND SYSTEM FOR CONSTRUCTING A DYNAMIC THREE-DIMENSIONAL MESH MODEL OF AN ANATOMICAL REGION

(71) Applicant: L&T TECHNOLOGY SERVICES LIMITED, Chennai (IN)

(72) Inventor: HAJA NAZURUDEEN MOHAMED SHEIK KATHI, Thanjavur (IN)

(21) Appl. No.: 19/319,775

(22) Filed: Sep. 5, 2025

(30) Foreign Application Priority Data
Dec. 24, 2024 (IN) 202441102571

(51) Int. Cl. G06T 17/20; G06T 7/149; G16H 30/40

(52) U.S. Cl. CPC G06T 17/20; G06T 7/149; G16H 30/40; G06T 2210/41

(57) ABSTRACT

A method for constructing a dynamic 3D mesh model of an anatomical region is disclosed. The method includes constructing a 3D volume of the anatomical region based on a plurality of DICOM image slices of the anatomical region and a verified 3D mesh model of the anatomical region. The method further includes segmenting, based on the verified 3D mesh model, the 3D volume to generate one or more segmented volumes using a deep-learning based volume segmentation model. The method further includes constructing a dynamic 3D mesh model of the anatomical region based on the one or more segmented volumes. The dynamic 3D mesh model is indicative of structural and functional characteristics of the anatomical region.

### TECHNICAL FIELD

This disclosure relates generally to image processing, and more particularly to method and system for constructing a dynamic three-dimensional mesh model of an anatomical region.

### BACKGROUND

Today, DICOM (Digital Imaging and Communications in Medicine) has emerged as a standard format for storing and transmitting medical images. However, the true potential of DICOM goes beyond mere 2D images. With advancements in technology, the conversion of DICOM data into 3D structures have revolutionized medical visualization, leading to enhanced diagnostics, precise treatment planning, and surgical precision. The conversion of the DICOM images into the 3D structures enhances collaboration and communication among healthcare professionals.

In present state of art, techniques for converting DICOM images into other image formats exist. Additionally, the techniques for constructing and segmenting 3D volumes of an anatomical region based on DICOM images may also exist. However, the existing techniques fail to convert the DICOM images into multi-dimensional immersive and interactive volumes efficiently and accurately.

The present invention is directed to overcome one or more limitations stated above or any limitations associated with the known arts.

### SUMMARY

In one embodiment, a method for constructing a dynamic three-dimensional (3D) mesh model of an anatomical region is disclosed. In one example, the method may include constructing a 3D volume of the anatomical region based on a plurality of DICOM image slices of the anatomical region and a verified 3D mesh model of the anatomical region. The method may further include segmenting, based on the verified 3D mesh model, the 3D volume to generate one or more segmented volumes using a deep-learning based volume segmentation model. It should be noted that each of the one or more segmented volumes may include an anatomical structure of the anatomical region. It should also be noted that the deep-learning based volume segmentation model is trained, using historical data, to identify the anatomical structure and segment the 3D volume based on the identified anatomical structure. The method may further include constructing a dynamic 3D mesh model of the anatomical region based on the one or more segmented volumes. It should be noted that the dynamic 3D mesh model is indicative of structural and functional characteristics of the anatomical region.

In another embodiment, a system for constructing a dynamic 3D mesh model of an anatomical region is disclosed. In one example, the system may include a processor and a computer-readable medium communicatively coupled to the processor. The computer-readable medium may store processor-executable instructions, which, on execution, may cause the processor to construct a 3D volume of the anatomical region based on a plurality of DICOM image slices of the anatomical region and a verified 3D mesh model of the anatomical region. The processor-executable instructions, on execution, may further cause the processor to segment, based on the verified 3D mesh model, the 3D volume to generate one or more segmented volumes using a deep-learning based volume segmentation model. It should be noted that each of the one or more segmented volumes may include an anatomical structure of the anatomical region. It should also be noted that the deep-learning based volume segmentation model is trained, using historical data, to identify the anatomical structure and segment the 3D volume based on the identified anatomical structure. The processor-executable instructions, on execution, may further cause the processor to construct a dynamic 3D mesh model of the anatomical region based on the one or more segmented volumes. It should be noted that the dynamic 3D mesh model is indicative of structural and functional characteristics of the anatomical region.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 illustrates a functional block diagram of an exemplary system for constructing a dynamic three-dimensional (3D) mesh model of an anatomical region, in accordance with some embodiments of the present disclosure.

FIGS. 2A and 2B illustrate a flow diagram of an exemplary process for constructing a dynamic 3D mesh model of an anatomical region, in accordance with some embodiments of the present disclosure.

FIGS. 3A and 3B illustrate a flow diagram of a detailed exemplary process for constructing a dynamic 3D mesh model of an anatomical region, in accordance with some embodiments of the present disclosure.

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

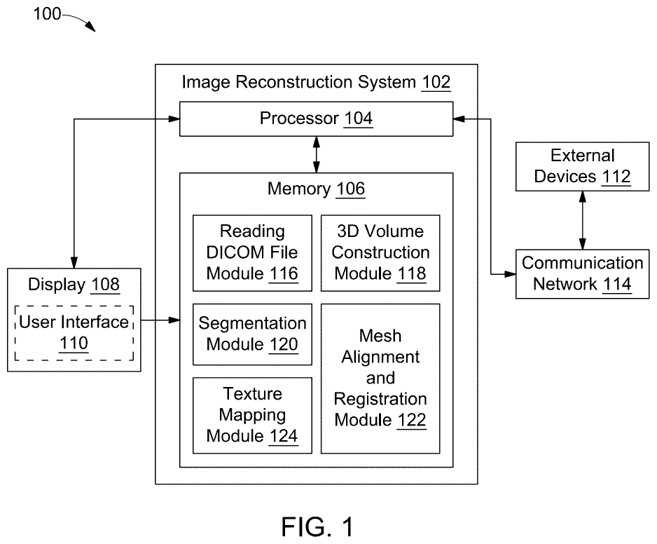

Referring now to FIG. 1, a functional block diagram of a system 100 for constructing a dynamic three-dimensional (3D) mesh model is illustrated, in accordance with some embodiment of the present disclosure. The system 100 may include an image reconstruction system 102. The image reconstruction system 102 may construct a dynamic 3D mesh model of an anatomical region. The dynamic 3D mesh model is an indicative of structural and functional characteristics of the anatomical region. The anatomical region may be, for example, but may not be limited to, head, neck, torso, upper extremities, and lower extremities.

The image reconstruction system 102 may include one or more processors 104, and a memory 106. The memory 106 may be a non-volatile memory (e.g., flash memory, Read Only Memory (ROM), Programmable ROM (PROM), Erasable PROM (EPROM), Electrically EPROM (EEPROM) memory, etc.) or a volatile memory (e.g., Dynamic Random Access Memory (DRAM), Static Random-Access memory (SRAM), etc.). The memory 106 may store instructions that, when executed by the one or more processors 104, may cause the one or more processors 104 to construct the dynamic 3D mesh model of the anatomical region, in accordance with aspects of the present disclosure.

The memory 106 may include a Digital Imaging and Communication in Medicine (DICOM) file reading module 116, a 3D volume construction module 118, a segmentation module 120, a mesh alignment and registration module 122, and a texture mapping module 124. Initially, the DICOM file reading module 116 may extract metadata from each of a plurality of DICOM image slices (i.e., DICOM files). By way of an example, the DICOM image files may include, but may not be limited to, CT scan files, MRI image files, X-Ray image files, ultrasound image files, etc. The plurality of DICOM image slices may include images of one or more anatomical structures. It should be noted that the metadata is indicative of at least one of structural and functional characteristics. The metadata may include at least one of a patient orientation, a slice thickness, a slice spacing, one or more imaging parameters, one or more spatial coordinates, a spatial alignment, a field of view (FOV), an image resolution. The DICOM file reading module 116 may employ a standard DICOM parser to extract key metadata fields.

The DICOM file reading module 116 may ensure the integrity of the metadata by verifying the completeness and consistency across the DICOM files. The DICOM file reading module 116 may flag any discrepancies or missing data in the metadata for further review. In other words, the DICOM file reading module 116 may parse DICOM headers for metadata fields, verify metadata integrity, and store metadata for use in subsequent steps.

Further, upon extracting the metadata, the DICOM file reading module 116 may analyze (or check) an order and an alignment of the plurality of DICOM image slices by utilizing a spatial registration technique to ensure that the plurality of DICOM image slices may be correctly sequenced and spatially aligned for accurate 3D reconstruction (the 3D reconstruction may be analogous to a 3D volume). To check the order and the alignment of the plurality of DICOM image slices, the DICOM file reading module 116 may compare the metadata with corresponding reference data from a verified 3D mesh model. In particular, the DICOM file reading module 116 may compare the spatial coordinates and align the plurality of DICOM image slices sequentially.

Further, upon comparison, if the DICOM file reading module 116 may detect any misalignment (or incorrect order) among the plurality of DICOM image slices, then the DICOM file reading module 116 may adjust the plurality of DICOM image slices based on comparison. The adjusting may include at least one of reordering a sequence of one or more of the plurality of DICOM image slices, correcting a spatial alignment of one or more of the plurality of DICOM image slices, correcting a spatial orientation of one or more of the plurality of DICOM image slices, adjusting a slice thickness of one or more of the plurality of DICOM image slices, interpolating one or more missing slices based on surrounding slices from the plurality of DICOM image slices, or refining one or more corrupted slices based on surrounding slices from the plurality of DICOM image slices.

To adjust the plurality of DICOM image slices, the DICOM file reading module 116 may extract pixel data from each of the plurality of DICOM image slices. Further, upon extracting the pixel data, the DICOM file reading module 116 may process the pixel data across the plurality of DICOM image slices based on the verified 3D mesh model to normalize intensity levels of the pixel data. In particular, the DICOM file reading module 116 may decode each of the pixel data extracted from the plurality of DICOM image slices. Further, the DICOM file reading module 116 may perform the pre-processing (such as the intensity normalization) to ensure that the pixel data is uniform and ready for further processing, particularly in terms of the intensity values and resolution.

Further, the DICOM file reading module 116 may interpret the pixel data in the context of the extracted metadata. This may ensure that the plurality of DICOM image slices may be correctly understood and ready for account factors (such as orientation, calibration, or any necessary corrections) based on the metadata. The DICOM file reading module 116 may use the extracted metadata to guide the interpretation of pixel data. Further, the DICOM file reading module 116 may adjust the plurality of DICOM image slices for orientation, calibration, and other critical factors to ensure that the plurality of DICOM image slices is correctly represented. It should be noted that, for validation, the DICOM file reading module 116 may compare the interpreted data against known standards (i.e., verified 3D mesh model) to flag any discrepancies for review.

Further, the 3D volume construction module 118 may construct a 3D volume of the anatomical region based on the plurality of DICOM image slices of the anatomical region and the verified 3D mesh model of the anatomical region. To construct the 3D volume, the 3D volume construction module 118 may sort and align the plurality of DICOM image slices based on the verified 3D mesh model to maintain a continuity of an anatomical structure and a spatial relationship among the plurality of DICOM image slices.

In particular, the 3D volume construction module 118 may sort the plurality of DICOM image slices based on their spatial coordinates or acquisition sequence. The sorting may ensure that the plurality of DICOM image slices may be arranged in the correct order to maintain the anatomical structure and spatial relationship required for the accurate 3D volume construction.

Further, upon sorting, the 3D volume construction module 118 may validate the spatial relationship between the plurality of DICOM image slices to ensure that the plurality of DICOM image slices may be correctly positioned relative to each other. Additionally, the 3D volume construction module 118 may correct any spatial discrepancies that may affect the 3D volume construction.

Further, upon validation, the 3D volume construction module 118 may align the plurality of DICOM image slices using the spatial registration technique to ensure that the plurality of DICOM image slices may be correctly positioned for the 3D volume construction.

Further, once the plurality of DICOM image slices is aligned, the 3D volume construction module 118 may stack the plurality of DICOM image slices upon sorting and aligning to construct the 3D volume. In other words, the 3D volume construction module 118 may stack the plurality of DICOM image slices to ensure that the constructed 3D volume may be accurately represent as the original anatomical structure. Further, once the plurality of DICOM image slices is stacked, the 3D volume construction module 118 may validate an alignment of the 3D volume based on the metadata extracted from each of the plurality of DICOM image slices. In other words, the 3D volume construction module 118 may cross-references the alignment of the plurality of DICOM image slices with the metadata to ensure that the plurality of DICOM image slices may be correctly aligned according to the imaging parameters and other relevant metadata.

Further, once the 3D volume is constructed, the segmentation module 120 may segment, based on the verified 3D mesh model, the 3D volume to generate one or more segmented volumes using a deep-learning based volume segmentation model. It should be noted that each of the one or more segmented volumes may include an anatomical structure of the anatomical region. The deep-learning based volume segmentation model is trained, using historical data, to identify the anatomical structure and segment the 3D volume based on the identified anatomical structure.

By way of an example, the segmentation module 120 may segment cardiac structures from the constructed 3D volume of a heart by isolating relevant anatomical features for further analysis and processing. For the segmentation, the segmentation module 120 may employ the deep learning-based techniques (such as the deep-learning based volume segmentation model) to accurately segment the cardiac structures from the 3D volume to handle variations in the image (i.e., DICOM image slices) quality and anatomical differences.

Further, upon generating the one or more segmented volumes, the segmentation module 120 may validate a quality and a consistency of each of the one or more segmented volumes for an artifact or for a variation in the anatomical structure. In case, the segmentation module 120 may detect any artifacts, or noise in one or more segmented volumes, then the segmentation module 120 may process each of the one or more segmented volumes to remove the artifact and smoothen the variation. In other words, the segmentation module 120 may post-process to refine each of the one or more segmented volumes to ensure high quality and accurate representation of the anatomical structures through smoothing, an artifact removal, or any other enhancements.

Further, the mesh alignment and registration module 122 may construct an initial 3D mesh model (i.e., the 3D mesh model, or dynamic 3D mesh model) of the anatomical region based on the one or more segmented volumes. By way of an example, the mesh alignment and registration module 122 may use the cardiac anatomical mesh geometry model to construct the 3D mesh model to ensure that the 3D mesh model is accurately reflects the anatomical features of the heart and maintains spatial relationships and structural integrity.

Further, the mesh alignment and registration module 122 may iteratively align the initial 3D mesh model with the plurality of DICOM image slices using spatial registration to generate an aligned 3D mesh model. In other words, the mesh alignment and registration module 122 may conduct a coarse alignment of the initial 3D mesh model using the spatial registration techniques for positioning the initial 3D mesh model close to the original anatomical structures to generate the aligned 3D mesh model.

Further, once the aligned 3D mesh model is generated, the mesh alignment and registration module 122 may validate the aligned 3D mesh model based on the verified 3D mesh model to generate a validated 3D mesh model. In particular, the mesh alignment and registration module 122 may evaluate the closeness of the initial alignment to determine if the aligned 3D mesh model is close enough to the original anatomy for a fine registration. If not, further adjustments are made.

Further, the mesh alignment and registration module 122 may adjust an alignment of the validated 3D mesh model based on the plurality of DICOM image slices to generate the dynamic 3D mesh model. In particular, the fine registration may align the 3D mesh model more precisely with the original plurality of DICOM image slices. The mesh alignment and registration module 122 may use the advanced spatial registration techniques to refine the alignment of the 3D mesh model and make precise adjustments to closely match the original anatomical structures in the plurality of DICOM image slices.

Further, the mesh alignment and registration module 122 may verify the anatomical accuracy of the aligned 3D mesh model to ensure that the aligned 3D mesh model may be correctly representing the anatomical structures before final validation.

Further, the mesh alignment and registration module 122 may conduct through validation of the aligned 3D mesh model to ensure that it meets predefined accuracy and quality standards. Additionally, confirming its suitability for further analysis or visualization. Further, the mesh alignment and registration module 122 may perform a detailed analysis to ensure that the alignment of the 3D mesh model with the plurality of DICOM image slices is accurate. Additionally, the mesh alignment and registration module 122 may make suitable adjustments in the 3D mesh model, if required.

Further, once the dynamic 3D mesh model is generated, the texture mapping module 124 may render the dynamic 3D mesh model for at least one of a visualization, a diagnostic analysis, or a surgical planning. The texture mapping module 124 may map textures derived from the plurality of DICOM image slices onto the dynamic 3D mesh model. In particular, the texture mapping module 124 may project the two-dimensional (2D) DICOM image slices textures onto the dynamic 3D mesh model to ensure that the textures may correspond accurately to the anatomical features. Further, upon texture projection, the texture mapping module 124 may validate the correspondence between the projected textures and the 3D mesh model to ensure the textures may be correctly aligned with the anatomical structures.

Further, the texture mapping module 124 may analyze the textures and the dynamic 3D mesh model to determine tissue characteristics using a machine learning model. In other words, the texture mapping module 124 may analyze the texture data to distinguish between different tissue types to ensure the accurate mapping of tissue characteristics in the original plurality of DICOM image slices. Further, the texture mapping module 124 may validate (or check) the texture-mapping of the 3D mesh model against the anatomical standards to ensure that the textures are correctly represents the original anatomical structures which may be crucial for the fidelity of the final 3D mesh model.

Further, upon validation, the texture mapping module 124 may finalize (or complete) the texture mapping process by applying final textures to the 3D mesh model to ensure smooth and accurate mapping. Further, the texture mapping module 124 may perform a final check to ensure that the texture-mapped 3D mesh model may accurately represent the original anatomical structures. Additionally, texture mapping module 124 may confirm that the 3D mesh model is ready for medical applications.

The system 100 may further include a display 108. The system 100 may interact with a user interface 110 accessible via the display 108. The system 100 may also include one or more external devices 112. In some embodiments, the image reconstruction system 102 may interact with the one or more external devices 112 over a communication network 114 for sending or receiving various data. The communication network 114 may include, for example, but may not be limited to, a wireless fidelity (Wi-Fi) network, a light fidelity (Li-Fi) network, a local area network (LAN), a wide area network (WAN), a metropolitan area network (MAN), a satellite network, the internet, a fiber optic network, a coaxial cable network, an infrared (IR) network, a radio frequency (RF) network, and a combination thereof. The one or more external devices 112 may include, but may not be limited to, a remote server, a laptop, a netbook, a notebook, a smartphone, a mobile phone, a tablet, or any other computing device.

It should be noted that all such aforementioned modules 116-124 may be represented as a single module or a combination of different modules. Further, as will be appreciated by those skilled in the art, each of the modules 116-124 may reside, in whole or in parts, on one device or multiple devices in communication with each other. In some embodiments, each of the modules 116-124 may be implemented as dedicated hardware circuit comprising custom application-specific integrated circuit (ASIC) or gate arrays, off-the-shelf semiconductors such as logic chips, transistors, or other discrete components. Each of the modules 116-124 may also be implemented in a programmable hardware device such as a field programmable gate array (FPGA), programmable array logic, programmable logic device, and so forth. Alternatively, each of the modules 116-124 may be implemented in software for execution by various types of processors (e.g., processor 104). An identified module of executable code may, for instance, include one or more physical or logical blocks of computer instructions, which may, for instance, be organized as an object, procedure, function, or other construct. Nevertheless, the executables of an identified module or component need not be physically located together but may include disparate instructions stored in different locations which, when joined logically together, include the module and achieve the stated purpose of the module. Indeed, a module of executable code could be a single instruction, or many instructions, and may even be distributed over several different code segments, among different applications, and across several memory devices.

As will be appreciated by one skilled in the art, a variety of processes may be employed for constructing a dynamic 3D mesh model of an anatomical region. For example, the image reconstruction system 102, may construct the dynamic 3D mesh model of the anatomical region, by the processes discussed herein. In particular, as will be appreciated by those of ordinary skill in the art, control logic and/or automated routines for performing the techniques and steps described herein may be implemented by the image reconstruction system 102 either by hardware, software, or combinations of hardware and software. For example, suitable code may be accessed and executed by the one or more processors on the image reconstruction system 102 to perform some or all of the techniques described herein. Similarly, application specific integrated circuits (ASICs) configured to perform some or all of the processes described herein may be included in the one or more processors on the image reconstruction system 102.

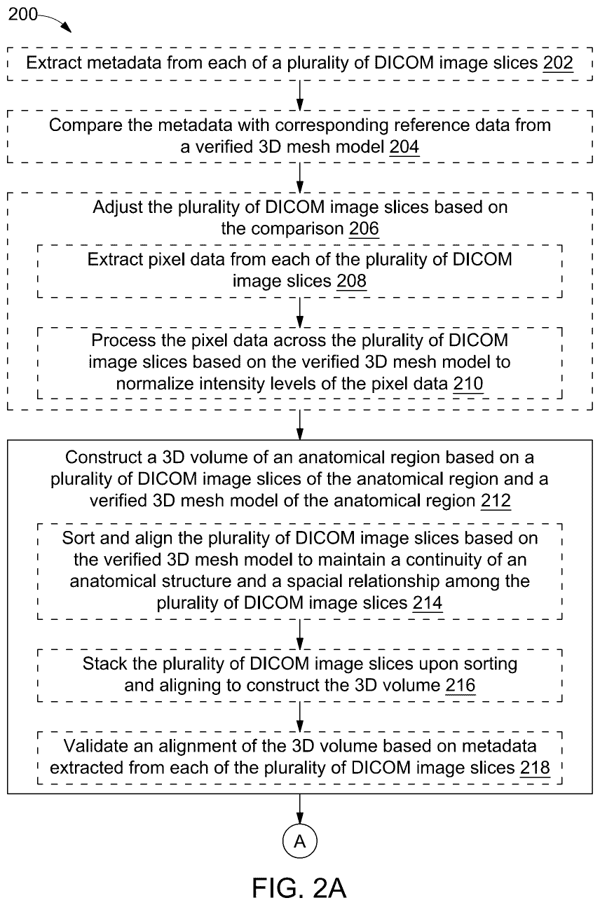

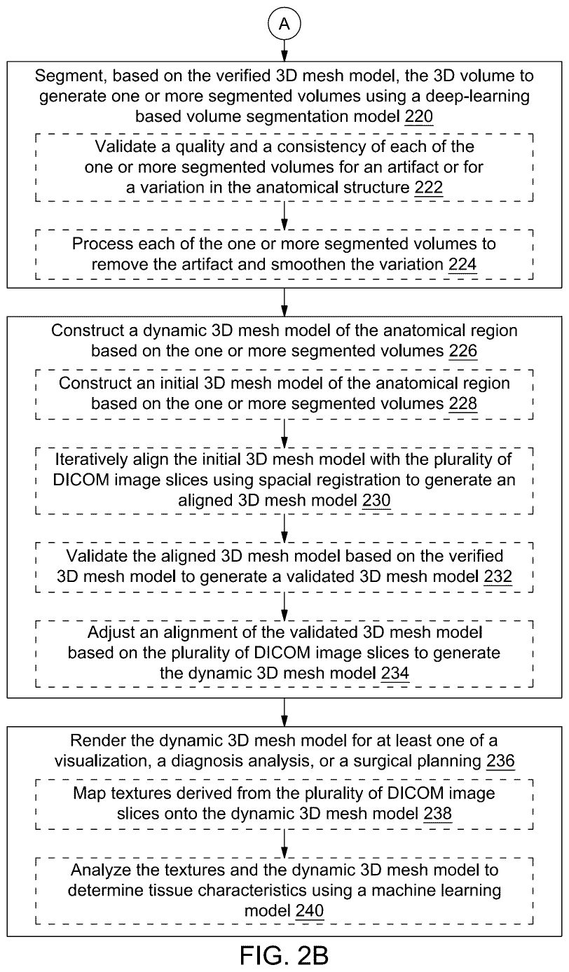

Referring now to FIG. 2A and FIG. 2B, an exemplary process 200 for constructing a dynamic 3D mesh model of an anatomical region is illustrated via a flow chart, in accordance with some embodiments of the present disclosure. FIG. 2A and FIG. 2B are explained in conjunction with FIG. 1. The process 200 may be implemented by the image reconstruction system 102 of the system 100. In some embodiments, the process 200 may include extracting, by a DICOM file reading module (such as the DICOM file reading module 116), metadata from each of a plurality of DICOM image slices, at step 202. Upon extracting the metadata, the process 200 may include comparing, by the DICOM file reading module, the metadata with corresponding reference data from a verified 3D mesh model, at step 204. It should be noted that the metadata is indicative of at least one of structural and functional characteristics and may include at least one of a patient orientation, a slice thickness, a slice spacing, one or more imaging parameters, one or more spatial coordinates, a special alignment, a filed of view, an image resolution.

Further, upon comparison, the process 200 may include adjusting, by the DICOM file reading module, the plurality of DICOM image slices based on the comparison, at step 206. It should be noted that adjusting may include at least one of reordering a sequence of one or more of the plurality of DICOM image slices, correcting, a spatial alignment of one or more of the plurality of DICOM image slices, correcting a spatial orientation of one or more of the plurality of DICOM image slices, adjusting a slice thickness of one or more of the plurality of DICOM image slices, interpolating one or more missing slices based on surrounding slices from the plurality of DICOM image slices, or refining one or more corrupted slices based on surrounding slices from the plurality of DICOM image slices.

To adjust the plurality of DICOM image slices, the step 206 of the process 200 may include extracting, by the DICOM file reading module, pixel data from each of the plurality of DICOM image slices, at step 208. Upon extracting the pixel data, the step 206 of the process 200 may include processing, by the DICOM file reading module, the pixel data across the plurality of DICOM image slices based on the verified 3D mesh model to normalize intensity levels of the pixel data, at step 210.

Further, the process 200 may include constructing, by a 3D volume construction module (such as the 3D volume construction module 118), a 3D volume of the anatomical region based on the plurality of DICOM image slices of the anatomical region and the verified 3D mesh model of the anatomical region, at step 212. To construct the 3D volume, the step 212 of the process 200 may include sorting and aligning the plurality of DICOM image slices based on the verified 3D mesh model to maintain a continuity of an anatomical structure and a spatial relationship among the plurality of DICOM image slices, at step 214. Further, the step 212 of the process 200 may include stacking, by the 3D volume construction module, the plurality of DICOM image slices upon sorting and aligning to construct the 3D volume, at step 216. Further, once the 3D volume is constructed, the step 212 of the process 200 may include validating, by the 3D volume construction module, an alignment of the 3D volume based on the metadata extracted from each of the plurality of DICOM image slices, at step 218.

Once the 3D volume is constructed, the process 200 may include segmenting, by a segmenting module (such as the segmenting module 120), the 3D volume to generate one or more segmented volumes using a deep-learning based volume segmentation model, at step 220. It should be noted that each of the one or more segmented volumes may include an anatomical structure of the anatomical region. It should also be noted that the deep-learning based volume segmentation model is trained, using historical data, to identify the anatomical structure and segment the 3D volume based on the identified anatomical structure.

Further, upon generating the one or more segmented volumes, the process 200 may include validating, by the segmenting module, a quality and a consistency of each of the one or more segmented volumes for an artifact or for a variation in the anatomical structure, at step 222. Upon successful validation, the process 200 may include processing, by the segmenting module, each of the one or more segmented volumes to remove the artifact and smoothen the variation, at step 224.

Further, the process 200 may include constructing, by a mesh alignment and registration module (such as the mesh alignment and registration module 122), a dynamic 3D mesh model of the anatomical region based on the one or more segmented volumes, at step 226. The dynamic 3D mesh model is indicative of structural and functional characteristics of the anatomical region. To construct the dynamic 3D mesh model, the process 200 may include constructing, by the mesh alignment and registration module, an initial 3D mesh model of the anatomical region based on the one or more segmented volumes, at step 228. Upon constructing the 3D mesh model, the process 200 may include iteratively aligning, by the mesh alignment and registration module, the initial 3D mesh model with the plurality of DICOM image slices using spatial registration to generate an aligned 3D mesh model, at step 230.

Further, upon generating the aligned 3D mesh model, the process 200 may include validating, by the mesh alignment and registration module, the aligned 3D mesh model based on the verified 3D mesh model to generate a validated 3D mesh model, at step 232. Further, upon generating the validated 3D mesh model, the process 200 may include adjusting, by the mesh alignment and registration module, alignment of the validated 3D mesh model based on the plurality of DICOM image slices to generate the dynamic 3D mesh model, at step 234.

Once the dynamic 3D mesh model is constructed, the process 200 may include rendering, by a texture mapping module (such as the texture mapping module 124), the dynamic 3D mesh model for at least one of a visualization, a diagnosis analysis, or a surgical planning, at step 236. The step 236 of the process 200 may include mapping, by the texture mapping module, textures derived from the plurality of DICOM image slices onto the dynamic 3D mesh model, at step 238. Further, the step 236 of the process 200 may include analyzing, by the texture mapping module, the textures and the dynamic 3D mesh model to determine tissue characteristics using a machine learning model, at step 240.

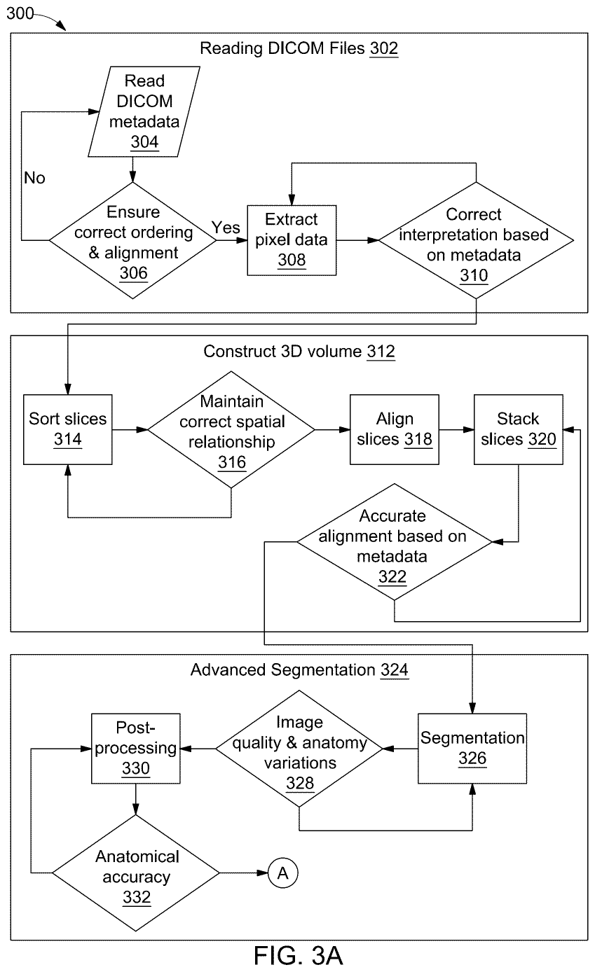

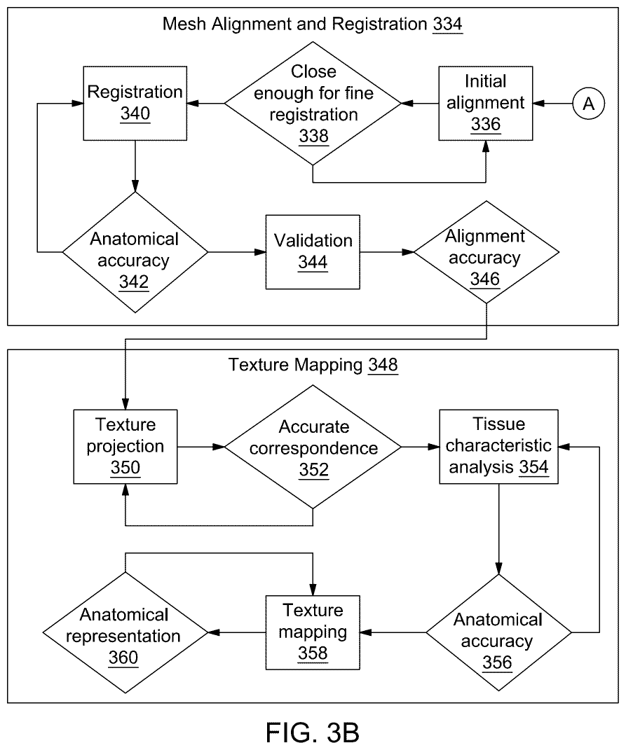

Referring now to FIG. 3A and FIG. 3B, a detailed exemplary process 300 for constructing a dynamic 3D mesh model of an anatomical region is illustrated via a flow chart, in accordance with some embodiments of the present disclosure. The process 300 may be implemented by the image reconstruction system 102 of the system 100. FIG. 3A and FIG. 3B are explained in conjunction with FIGS. 1 and 2. In an embodiment, the process 300 may include reading DICOM files (e.g., CT scan files, MRI image files, X-Ray image files, ultrasound image files, etc.), at step 302. The step 302 of the process 300 may include steps 304, 306, 308, and 310.

The process 300 may include reading DICOM metadata, at step 304. Initially, the DICOM file reading module 116 may receive DICOM images as an input (such as the plurality of DICOM image slices). Upon receiving the DICOM images, the DICOM file reading module 116 may extract metadata from the DICOM images using a DICOM parser algorithm. The metadata is indicative of at least one of structural and functional characteristics. The metadata may include at least one of a patient orientation, a slice thickness, a slice spacing, one or more imaging parameters, one or more spatial coordinates, a spatial alignment, a field of view, an image resolution. It should be noted that the DICOM file reading module 116 may flag any discrepancies or missing data using the DICOM parser algorithm for further review.

By way of an example, an exemplary metadata extracted from their respective DICOM images for some anatomical regions is described below.

For cardiac anatomical region, the metadata may include information, such as the patient's heart rate and slice acquisition parameters. This data may help in timing cardiac cycles for dynamic studies and adjusting measurements accordingly.

For brain anatomical region, the metadata may specify voxel size and patient positioning which may be crucial for accurately measuring brain structures and analyzing symmetry (e.g., as in cases of trauma or hydrocephalus).

For breast anatomical region, the metadata may include information, such as breast density and scan orientation. The metadata may help to assess how tissue density may affect visibility and interpretation of potential tumours.

Further, once the metadata is extracted, the DICOM file reading module 116 may compare the metadata with corresponding reference data from the verified 3D mesh model. It should be noted that the verified 3D mesh model may be analogous to the 3D mesh, or a clinically verified 3D mesh geometry. In other words, the metadata (such as the patient orientation, the slice thickness and the like) may be compared with anatomical details from the 3D mesh.

To compare the metadata with the corresponding reference data from the clinically verified 3D mesh geometry, the DICOM file reading module 116 may align imaging parameters and the metadata extracted from the DICOM images with the (reference) anatomical details received from the clinically verified 3D mesh geometry (i.e., the verified 3D mesh model). The comparison may ensure that the DICOM images are correctly oriented, spaced, and aligned to match the anatomical reality represented by the clinically verified 3D mesh geometry. This step reduces errors and ensures that subsequent processes, such as segmentation and 3D reconstruction, yield clinically reliable results.

By way of an example, the patient orientation from the DICOM metadata (such as the metadata) may be compared with the expected orientation of the anatomical structure in the clinically verified 3D mesh geometry. Such comparison may ensure that the DICOM images may be oriented correctly relative to the anatomical planes of the body (such as left-right, anterior-posterior). This may be crucial for interpreting the images (analogous to a plurality of DICOM image slices) consistently and for correctly aligning the image slices with the clinically verified 3D mesh geometry.

In a similar manner, the slice thickness and inter-slice spacing from the DICOM metadata may be compared with the expected anatomical spacing in the clinically verified 3D mesh geometry to verify that the image slices may have appropriate and consistent thickness which is necessary for accurately reconstructing the 3D volume.

Imaging parameters (e.g., heart rate for a cardiac imaging) may be compared with the dynamic nature of the clinically verified 3D mesh geometry (which, in case of cardiac region, represents the anatomy of the heart at different phases of the cardiac cycle). Such comparison may ensure that the timing of the DICOM images may correspond to the right phase of the cardiac cycle for improving the accuracy of functional assessment (such as ejection fraction, or wall motion analysis). It should be noted that in cardiac imaging, the heart rate metadata may be crucial to determine which phase of the heart cycle is captured. This is essential for assessing dynamic features (e.g., valve movement).

In a similar manner, the spatial coordinates from the DICOM metadata may be compared with the clinically verified 3D mesh geometry to ensure that structures (e.g., heart chambers, brain lobes, or breast tissue) may be correctly aligned. The spatial coordinates comparison may be performed to maintain the anatomical consistency and avoid misalignment issues that may affect measurements (e.g., chamber volumes in the heart, tumor size in the brain or breast), and the field of view (FOV) and resolution from the DICOM images may be compared with the expected anatomical detail in the clinically verified 3D mesh geometry to ensure that the image slices may cover the necessary anatomical structures and may provide enough resolution for an accurate segmentation and analysis.

The step 304 may be performed to get ensure that the anatomical orientation may be interpreted correctly and provide context for the images. By way of an example, in cardiac analysis, it ensures that the heart's orientation and size correspond to clinically accepted standards which are crucial for accurate subsequent steps.

Further, upon comparison, the process 300 may include ensuring correct order and alignment of the DICOM images, at step 306. The DICOM slices and the clinically verified 3D mesh geometry may be received as an input. To ensure the correct order and alignment of the DICOM images, the DICOM file reading module 116 may check and adjust the order and alignment of the DICOM images slices using the clinically verified 3D mesh geometry. By way of an example, in cardiac imaging, the step 306 may ensure that the structures (e.g., the heart chambers and valves) may be properly aligned and sequenced. The step 306 may be performed to ensure correct ordering and alignment preserves anatomical fidelity. This may be essential for reconstructing an accurate 3D model (analogous to the 3D volume) which is critical for analysing blood flow or structural abnormalities in the heart, brain, or breast. A spatial registration algorithm may be used to align and order the DICOM image slices based on spatial coordinates to ensure that the anatomical structures may be arranged correctly.

In continuation with the above example, for the cardiac, proper ordering and alignment of heart DICOM image slices allow for accurate assessment of the flow of blood through the heart chambers. On the other hand, if there is any misalignment that may be detected in the heart DICOM image slices, then the misalignment corrections may be crucial for measuring the size and function of heart valves.

For the brain, correct order may be essential for accurately visualizing continuous structures (such as a brainstem or a ventricular system) which may be disrupted by tumors or hemorrhage.

For the breast, proper alignment may ensure accurate spatial representation of glandular tissue, aiding in assessing the spread or margins of suspicious lesions.

At step 306, the anatomical consistency and alignment of the DICOM images may be analyzed. For example, in cardiac imaging, this step ensures that the heart chambers may be properly aligned and setting the foundation for measuring chamber volumes and wall thickness later.

Further, the process 300 may include extracting pixel data, at step 308. The pixel data may be extracted from the DICOM image slices using a pixel extraction and normalization algorithm. It should be noted that the clinically verified 3D mesh geometry may be used as a reference model to enhance an analysis of the pixel data extracted from the DICOM image slices. The clinically verified 3D mesh geometry may provide a spatial accurate representation of the anatomical structures. Upon extracting the pixel data, the process 300 may include analyzing the extracted pixel data based on the clinically verified 3D mesh model to normalize intensity levels of the pixel data using a segmentation algorithm. When analyzing the pixel data, then the segmentation algorithm may use this reference model (i.e., the clinically verified 3D mesh geometry) to improve the precision of boundaries between the different tissues. By way of an example, in cardiac imaging, the segmentation algorithm may help in accurately separating the myocardium from the surrounding tissues, while in the brain imaging, the segmentation algorithm may assist in clearly defining the boundaries of brain regions.

It should be noted that the clinically verified 3D mesh geometry may help to contextualize (or normalize) the intensity values of the pixel data by providing an anatomical landmark. By way of an example, in breast imaging, areas of higher intensity (which may indicate the dense tissue or calcifications) may be analysed in relation to the clinically verified 3D mesh geometry mesh to determine whether these features may align with the expected anatomical patterns or not for further processing.

By way of an example, for cardiac, the extracted pixel data from the DICOM image slices may contain various intensity levels corresponding to different tissues (such as, a myocardium, blood, and surrounding structures). Further, the clinically verified 3D mesh geometry may provide a detailed map of where these anatomical structures are located. Further, the extracted pixel data may be compared with the clinically verified 3D mesh geometry. Upon comparison, the DICOM file reading module 116 may identify the regions of interest (such as the myocardial tissue or coronary arteries) more accurately to differentiate between the heart's anatomical features and may focus the analysis on critical areas (such as detecting blockages in arteries).

For brain, the clinically verified 3D mesh geometry may help to distinguish between grey matter, white matter, and other regions (such as tumors or lesions). Additionally, the clinically verified 3D mesh geometry may be used as a reference model to highlight areas of interest, to ensure that the pixel intensity variations may be correctly mapped to specific brain structures. This is important for identifying abnormalities like tumors or atrophy.

For breast, differences in the pixel intensity highlight dense tissue and potential calcifications which are often early indicators of breast cancer.

Further, the process 300 may include correcting, by the DICOM file reading module 116, an interpretation based on the metadata, at step 310. The DICOM metadata and the extracted pixel data may be received as an input. The interpretation of the pixel data may be refined using the anatomical orientation and structure from the 3D mesh. For example, in cardiac imaging, the 3D mesh may help to align heart structures for accurate wall thickness measurements. The step 310 may be performed to ensure the DICOM images may be interpreted correctly and may adjust for patient orientation and slice thickness, if necessary. Accurate interpretation is crucial for diagnosing conditions (like myocardial infarction, brain tumours, or breast cancer). A metadata-based correction algorithm may be used to adjust images orientation and scale to ensure accurate anatomical representation in the final model.

In continuation with the above example, for cardiac, using metadata, images may be oriented properly to view cardiac structures. This may allow accurate assessment of wall thickness which may be important for diagnosing conditions (e.g., hypertrophic cardiomyopathy).

For brain, correct interpretation based on the metadata may ensure the correct orientation of brain images, helping in the measurement of structural asymmetries or the assessment of midline shifts due to tumours or trauma.

For breast, correct interpretation based on metadata may help to ensure that any detected masses are properly aligned, aiding in more accurate measurement and diagnosis.

At step 310, adjustments to the intensity values and orientation may be analyzed. By way of an example, in breast imaging, metadata ensures that tissue density may be interpreted correctly which is critical for identifying areas of concern (like dense tissue that could obscure tumours).

Further, the process 300 may include constructing, by the 3D volume construction module 118, a 3D volume, at step 312. The step 312 may include further steps 314, 316, 318, 320, and 322. Using the clinically verified 3D mesh geometry provides significant advantages that would be difficult to achieve without it. To enhance accuracy and consistency with the clinically verified 3D mesh geometry at step 312, the clinically verified 3D mesh geometry provides a gold standard reference for anatomical structures. It ensures that DICOM slices are correctly ordered and aligned based on real, clinically verified anatomical relationships. This helps maintain the integrity of complex structures like the heart chambers, brain lobes, or breast tissue, leading to highly accurate 3D reconstructions.

On the other hand, without using the clinically verified 3D mesh geometry, there would be no reliable way to validate the order and spatial alignment of the DICOM slices. Disordered or misaligned slices could lead to significant anatomical errors, such as incorrectly reconstructed organs or distorted features. This can be especially problematic in cases where the anatomy is highly complex, or the imaging conditions vary.

To reduce ambiguity in slice alignment at step 312 with the clinically verified 3D mesh geometry, the clinically verified 3D mesh geometry acts as a guide to correct any misalignment or distortion in the DICOM slices. It provides a clear reference to adjusting the position and orientation of slices, ensuring spatial consistency.

On the other hand, without using the clinically verified 3D mesh geometry, it would be challenging to detect and correct misalignments, especially when dealing with small variations in slice orientation. This could result in a 3D model that does not accurately represent the true anatomy, which could compromise diagnostic or research outcomes.

To facilitate complex structure analysis with the clinically 3D mesh geometry at step 312. For organs with intricate structures (like the heart or brain), the 3D mesh helps segment and analysed features more precisely. It assists in delineating boundaries, such as separating the grey and white matter in the brain or defining heart chamber walls.

On the other hand, without using the clinically 3D mesh geometry, segmentation and feature analysis would rely solely on the intensity and contrast in the DICOM images, which may not always be clear. Variability in image quality, noise, or overlapping structures could make accurate segmentation difficult, leading to errors in the analysis.

To handle anatomical variations with the clinically verified 3D mesh geometry at step 312. The mesh can account for variations in anatomy, such as differences in organ size, shape, or orientation between patients. It provides a robust reference for ensuring that these variations are captured accurately.

On the other hand, without using the clinically verified 3D mesh geometry, there would be no reference for handling anatomical variations, making it difficult to differentiate between normal and pathological features. This could result in misinterpretation of important diagnostic information.

Some of the challenges of not using the clinically verified 3D mesh geometry at the step 312 is described as below.
Increased Error Rate: Without the 3D mesh, there is a higher risk of errors in slice ordering and alignment, which can lead to incorrect 3D reconstructions.
Trust on Image Quality: The accuracy of segmentation and analysis would depend heavily on the quality of the DICOM images. Poor image quality or artifacts could severely affect the results.
Manual Corrections: In the absence of a 3D mesh, more manual intervention might be required to check and adjust the alignment of slices, increasing the time and complexity of the process.

The process 300 may include sorting, by the 3D volume construction module 118, the DICOM image slices, at step 314. The DICOM image slices and the 3D mesh may be received as an input. Further, the clinically verified 3D mesh geometry may be used as a reference to sort and organize the DICOM image slices properly. By way of an example, for the brain, sorting may ensure the correct arrangement of cortical and subcortical regions. Sorting the DICOM image slices may be necessary for building a coherent 3D model (analogous to a dynamic 3D mesh model). The step 314 may be performed to facilitate precise measurement of the cardiac chamber volumes, the brain structures, or the breast tissue masses. A sorting algorithm based on spatial coordinates may ensure that the DICOM image slices may be arranged in the correct order for volume reconstruction (analogous to the 3D volume).

By way of an example, for the cardiac, sorting the DICOM image slices may be necessary for creating a coherent 3D model of the heart. This enables the measurement of chamber volumes and the assessment of septal defects.

For brain, sorting the DICOM image slices may be crucial for examining the continuity of brain structures which is important for the tumor localization and understanding the extent of damage in stroke patients.

For breast, properly sorted the DICOM image slices may ensure that breast tissue is consistently represented, aiding in the assessment of tumour dimensions and the analysis of tissue density patterns.

The continuity and the order of the anatomical features may be analyzed, at step 314. By way of an example, for the cardiac imaging, sorting the DICOM image slices correctly helps in constructing a continuous representation of the heart, necessary for measuring the size and shape of the chambers.

Further, upon sorting the DICOM image slices, the process 300 may include maintaining, by the 3D volume construction module 118, a correct spatial relationship between the DICOM image slices, at step 316. The ordered DICOM image slices may be received as an input. Upon receiving the ordered DICOM image slices, the 3D volume construction module 118 may verify the spatial relationship between the DICOM image slices based on the clinically verified 3D mesh geometry. The step 316 may be performed to maintain the spatial relationship between the DICOM image slices which is essential for accurately reconstructing the 3D volume. It may ensure that the measurements (like septal defects in the heart or tumour spread in the brain and breast) may be reliable. A spatial relationship algorithm may be used to check the positioning of the DICOM image slices and may adjust the DICOM image slices if required to maintain the anatomical accuracy. In some embodiments, if the spatial relationship among the DICOM image slices may not be correctly maintained, then the step 314 may be repeated till the spatial relationship is correctly maintained.

By way of an example, for cardiac, ensuring the correct spatial relationship between the DICOM image slices may be important for accurately visualizing the heart chambers and major vessels. Additionally, it may be essential for diagnosing valvular and structural heart diseases.

For brain, this step may preserve the spatial arrangement of brain regions which may be vital for analyzing the effects of space-occupying lesions or for planning surgical interventions.

For breast, maintaining the spatial relationship may allow for accurate evaluation of the extent of a tumour and its relation to surrounding tissues.

The spatial positioning of structures may be analyzed, at step 316. By way of an example, in brain imaging, maintaining the spatial relationship between cortical structures is important for assessing abnormalities like brain atrophy or tumours displacement.

Further, once the spatial relationship between the DICOM image slices is verified, the process 300 may include aligning, by the 3D volume construction module 118, the DICOM image slices, at step 318. In some embodiments, when the DICOM image slices are stacked to form the 3D volume, in some cases, the DICOM image slices may have slight misalignments or distortions. This may happen due to patient movement, inconsistencies in image acquisition, or subtle variations in how the imaging equipment captured the DICOM image slices. The 3D mesh geometry serves as a benchmark (or reference model) to ensure that these issues may be corrected.

To correct the misalignments of the DICOM image slices, the 3D mesh which represents the anatomically accurate 3D structure of the organ may be used to guide adjustments in the alignment of the DICOM image slices. The alignment step 318 of the process 300 may match the anatomical features in the DICOM image slices to their corresponding features in the 3D mesh. For example, in breast imaging, the 3D mesh may help to ensure that tissue layers may be aligned smoothly and continuously, without distortions or overlaps. In some embodiments, in the breast imaging, the overlapping nature of glandular, fatty, and fibrous tissues may make it difficult to discern and align anatomical features correctly. Misalignment may lead to distortions which may obscure important diagnostic details.

To overcome this issue, the 3D mesh may provide a spatially accurate representation of how breast tissues should be arranged. It may act as a guide to separate and align these tissues correctly, reducing the risk of errors. By comparing the alignment of DICOM image slices with the 3D mesh. Further, the system may make the fine adjustments to ensure that the overlapping tissues may be accurately represented and distinguished.

It should be noted that using the 3D mesh to fine-tune the DICOM image slice alignment may ensure that the reconstructed volume is anatomically correct. This is crucial for accurate diagnosis and analysis, especially in regions with complex or overlapping tissue structures. Additionally, the 3D mesh may help to eliminate distortions that may affect the continuity of the anatomical features. This is particularly important for visualizing detailed structures (like ducts or masses in breast imaging).

The step 318 of the process 300 may include fine-tuning the alignment of slices to ensure that the anatomical structures may be continuous and undistorted. The spatially related DICOM image slices and the 3D mesh may be received as an input. By way of an example, in breast imaging, this step 318 may help in distinguishing overlapping tissues (as already explained above). The correct alignment of the DICOM image slices may facilitate a seamless 3D reconstruction (i.e., the 3D volume) which is necessary for assessing features (such as ventricular wall thickness in the heart or tumour boundaries in the brain). An affine and non-rigid registration algorithm may be employed to align DICOM image slices. Additionally, the Affine and non-rigid registration algorithms may also employ to correct distortions and maintain structural continuity.

In continuation with the above example, for the cardiac, the DICOM image slices alignment may ensure the continuity in heart structures and enable the precise measurement of the thickness of the ventricular walls and may detect the structural abnormalities.

For the brain, the DICOM image slices alignment may be critical for analyzing the brain cortex and subcortical structures. Misalignment correction allows for better assessment of lesions or the ventricles.

For the breast, proper DICOM image slices alignment may help to distinguish between the overlapping tissues and make it easier to identify and measure the abnormalities.

The step 318 may be performed to ensure the anatomical structures may be aligned properly. By way of an example, in cardiac imaging, the step 318 may be crucial to ensure the septum and ventricular walls are properly aligned for accurate wall thickness measurements.

Further, upon properly aligning the DICOM image slices, the process 300 may include stacking, by the 3D volume construction module 118, the DICOM image slices upon sorting and aligning to construct the 3D volume (analogous to the coherent 3D volume), at step 320. The 3D volume may be, for example, model of heart, brain, and breast. When stacking the 2D DICOM image slices to create the 3D volume it may be essential to maintain the correct spatial relationships between the anatomical structures. The clinically verified 3D mesh geometry may provide a detailed and anatomically accurate reference model which may represent how these anatomical structures should be aligned and positioned in 3D space. As each of the DICOM image slices may be added to the stack. Further, the 3D volume construction module 118 may compare the emerging 3D volume with the clinically verified 3D mesh geometry. By way of an example, the 3D mesh may guide the stacking process to ensure that the anatomical structures (such as heart chambers, brain lobes, or breast tissue layers) may be aligned and spaced correctly. This may reduce the risk of distortions or anatomical inaccuracies.

The DICOM image slices may be stacked to construct the 3D volume using the 3D mesh. It should be noted that the 3D volume may be required for detailed analysis (e.g., evaluating chamber sizes in the heart, tumours volume in the brain, or mass distribution in the breast). A volume stacking algorithm may be used to ensure that the DICOM image slices are arranged correctly to form the coherent 3D volume to preserve anatomical relationships.

In continuation with the above example, for cardiac, stacking the aligned DICOM image slices may form a 3D model of the heart. This may be used to measure chamber volumes, assess wall motion, and analyse septal defects.

For brain: A stacked 3D model of the brain may be used for volumetric analysis (such as calculating tumour volume or assessing brain atrophy).

For breast: A 3D representation of the breast allows for the assessment of tumour volume, shape, and the relationship with surrounding tissues. The step 320 may accurately construct the 3D volume for further processing.

At step 320, overall 3D volume reconstruction accuracy may be analyzed. By way of an example, in breast imaging, stacking slices accurately allows for volumetric analysis of masses, which is important for assessing tumours size and spread.

Further, upon constructing the 3D volume, the process 300 may include validating, by the 3D volume construction module 118, the accurate alignment of the 3D volume based on the metadata, at step 322. In an embodiment, when the DICOM image slices may be aligned on the 3D mesh to create the 3D volume, there may be some slight errors or discrepancies that occur in the positioning of the anatomical features due to variations in the image acquisition or patient movement. To overcome this problem, the reconstructed 3D volume may be compared with the 3D mesh to identify any misalignments. Further, necessary adjustments may be made if any misalignments may be detected. In a particular, the 3D volume construction module 118 may check whether the structures in the reconstructed 3D volume (such as the brain ventricles or coronary arteries) may be properly matched the expected anatomical layout or not using the 3D mesh. In case, if any features are out of place or incorrectly oriented, the 3D mesh may provide a guide for refining their alignment. By way of an example, in brain imaging, the 3D mesh may ensure that the brain ventricles may be properly aligned and symmetrically positioned which is critical for assessing conditions (like hydrocephalus or brain asymmetry). In some embodiments, differences in imaging parameters, patient positioning, or even slight distortions in the DICOM image slices may affect the accuracy of the reconstructed 3D volume. In such cases, the 3D mesh may serve as a consistent reference which may help to correct these variations and ensure that the anatomical structures may be in their true position. By way of an example, by ensuring that structures (like coronary arteries and brain ventricles) are correctly positioned, then the 3D mesh may help to create a reliable 3D model that may be used for accurate diagnosis and treatment planning. Using the 3D mesh may minimize the risk of alignment errors that could lead to misinterpretations of the anatomy or missed diagnoses.

The alignment is refined using metadata and compared to the 3D mesh to ensure accuracy. This ensures structures like coronary arteries or brain ventricles are correctly positioned. This step ensures that the reconstructed volume aligns with clinical standards, aiding in precise measurements and diagnostics. A metadata-driven alignment algorithms adjust the 3D volume to match imaging parameters and anatomical expectations.

In continuation with the above example, for cardiac, alignment may ensure the heart's orientation and dimensions may be accurate to allowing for the precise assessment of coronary artery blockages or heart valve function.

For brain, this step helps in accurately positioning the brain structures for comparison with standardized models, aiding in the detection of structural deviations.

For breasts, accurate alignment aids in determining the spatial characteristics of breast tumours, ensuring consistent measurement and localization.

Further, the process 300 may include advanced segmentation, at step 324. The step 324 of the process 300 may further include steps 326, 328, 330, and 332. The process 300 may include segmenting, by the segmentation module 120, the 3D volume, at step 326. The segmentation module 120 may segment the 3D volume (e.g., heart, chambers, brain tumours, or breast masses) to generate one or more segmented volumes (analogous to the segmented images, or segmented structures) using a deep-learning based volume segmentation model (analogous to the deep-learning based segmentation algorithm). The deep-learning based segmentation algorithms (e.g., convolutional neural networks (CNNs), and the like) may be used to accurately isolate and segment the anatomical features. Segmentation enables accurate identification and measurement of critical features (such as artery blockage in the heart, tumours boundaries in the brain, or dense tissue in the breast).

By way of an example, consider a heart. For the heart, the 3D mesh may contain a detailed representation of the heart's anatomy which may include the shapes and spatial relationships of structures (such as the left ventricle, right ventricle, atria, valves, and major blood vessels). During segmentation, the 3D mesh (i.e., the reference model) may help the algorithm (i.e., the deep-learning segmentation algorithm) to understand where each structure is supposed to be and how it is shaped. When segmenting the heart chambers, the 3D mesh may guide the algorithm to correctly outline the walls of the left and right ventricles, even if the DICOM images have areas with poor contrast or noise.

In some embodiments, the cardiac structures (like the heart muscle (or myocardium) may have fuzzy or unclear boundaries in their DICOM images. Further, upon detecting some fuzzy or unclear boundaries in the DICOM images, the 3D mesh may help the segmentation algorithm to distinguish between different tissues more accurately. It may ensure that the boundaries of the ventricles, atria, and valves may be precisely defined.

By way of an example, if the myocardium's boundary may be difficult to see in the DICOM images due to low contrast, then the 3D mesh may provide a guide that may help the segmentation algorithm to correctly segment the heart muscle. Additionally, the segmentation algorithm may ensure that the measurements (e.g., wall thickness) may be accurate.

It should be noted that the heart has intricate structures (such as the aortic valve or the interventricular septum) that may be challenging to segment correctly. In such cases, the 3D mesh may provide a reliable reference to minimize errors in these complex regions. Fo example, in cases where the DICOM images may be affected by artifacts or patient movement, the 3D mesh may help the segmentation algorithm to identify and correct segmentation errors and may ensure that the critical features (like the valves and septum) may be accurately captured.

In continuation with the above example, for cardiac, the segmentation may isolate heart chambers, valves, and arteries. Additionally, measurements like artery blockage percentage, ventricular wall thickness, and chamber size differences may be accurately obtained.

For brain, segmentation may separate different brain tissues which allow for the measurement of tumour boundaries, the extent of edema, and the volume of cerebrospinal fluid spaces.

For breast, the segmentation may identify dense tissue and suspicious masses. This step is crucial for measuring tumour dimensions and assessing the extent of malignancy.

Detailed tissue boundaries and shapes of the one or more segmented volumes may be analyzed, at step 326.

By way of an example, for heart, wall thickness, chamber size, artery blockage, and septal defects may be analyzed. For example, in cardiac imaging, the left ventricular wall thickness is measured to assess hypertrophy. Artery blockage may be identified by examining the coronary arteries for narrowing.

For brain, tumour boundaries (such as grey vs. white matter) and cerebrospinal fluid spaces may be analyzed. For example, in brain imaging, tumours may be segmented and measured for volume and location relative to critical structures.

For breast, mass margins, tissue density, and calcifications may be analyzed. For example, in breast imaging, the segmentation step allows for the identification and characterization of tumours, assessing whether they have irregular or smooth margins.

Further, upon generating the one or more segmented volumes, the process 300 may include verifying, by the segmented module 120, an image quality and anatomy variations of the one or more segmented volumes, at step 328. The one or more segmented volumes may be received as an input. The image quality may be assessed against the 3D mesh to identify artifacts or anatomical variations in the one or more segmented volumes. This may ensure the accurate representation of features like myocardial tissue or brain lesions. Quality control may reduce errors and ensure the reliable measurements which may be crucial for diagnosing heart disease, brain tumours, or breast cancer. An image quality assessment algorithm may be used to detect artifacts, and anatomical variation detection algorithms may be configured to compare the segmented structures to the standard models (i.e., 3D mesh).

In continuation with the above example, for cardiac, the quality of heart images is assessed, and anatomical variations like congenital defects may be noted. Wall thickness or chamber size inconsistencies may be flagged for further review.

For brain, image quality assessment may help to identify artifacts that may obscure critical areas like tumour margins or brainstem structures. Anatomical variations may be also documented.

For breast, image quality may be evaluated to ensure clarity in detecting calcifications or small tumours. Variations in breast tissue density may be noted for more accurate diagnostics.

At step 328, detection of the artifacts and the anatomical variation may be analyzed. For example, in brain imaging, the presence of edema or hemorrhage may be identified as variations in tissue characteristics.

Further, once the segmented images are assessed, the process 300 may include post-processing, by the segmentation module 120, the one or more segmented volumes, at step 330. The post-processing step may refine the segmentation boundaries of each of the one or more segmented volumes to enhance the anatomical accuracy using the 3D mesh. Post-processing may improve the model's accuracy and make it more suitable for clinical analysis or surgical planning. A Smoothing and artifact removal algorithm may be used to refine the edges of the segmented structures to enhance the overall image quality.

In continuation with the above example, for cardiac, post-processing step (i.e., the step 330) may smooth the boundaries and refine the segmentation of heart structures which may improve measurements of the ejection fraction or the size of aneurysms.

For brain, refinement may help in better delineating brain tumours and reducing noise, making measurements of tumour growth or cortical thinning more accurate.

For breast, the post-processing step may smooth the boundaries of detected masses, providing a clearer view for measuring tumour margins and assessing the likelihood of malignancy.

At the step 330, refinement of the segmentation boundaries may be analyzed. For example, in cardiac imaging, post-processing may smooth the contours of the heart chambers for accurate ejection fraction calculation.

Further, upon refining the segmented images, the process 300 may include constructing a detailed anatomical model (may be analogous to a dynamic 3D mesh model) using the 3D mesh, at step 332. In particular, the 3D mesh may be applied to construct the detailed anatomical model. The mesh-based 3D model allows for detailed visualization and analysis of anatomical features, supporting clinical applications like surgery or tumours treatment. A Mesh construction algorithm may be used to generate the 3D mesh model from the segmented images to ensure the anatomical accuracy.

In continuation with the above example, for cardiac, the segmented data may be used to create a detailed 3D mesh of the heart which may help in visualizing complex structures and assessing artery blockages or valve defects.

For brain, a 3D brain mesh allows for the simulation of brain surgeries or the analysis of tumour pressure on surrounding tissues.

For breast, a breast mesh may provide a 3D view of tumour(s), aiding in surgical planning or radiation therapy.

At step 332, the structural intensity of the heart features may be analyzed. For example, this step may enable the assessment of valve geometry and blood flow dynamics in the heart.

Further, upon constructing the 3D mesh model, the process 300 may include mesh alignment and registration of the constructed 3d mesh model, at step 334. The step 334 may include further steps, 336, 338, 340, 342, 344. The process 300 may include initially aligning, by the mesh alignment and registration module 122, the constructed 3D mesh model with the DICOM images, at step 336. The sets pf foundation for precise alignment may ensure that the 3D mesh model is ready for the fine adjustments. Coarse registration algorithms may be used to perform the initial alignment and positioning the 3D mesh close to the anatomical structures.

In continuation with the above example, for cardiac, initial alignment may position the heart mesh in relation to the original images. This step may be the starting point for detailed analysis of structures like the aortic root.

For brain, the brain mesh is coarsely aligned for a general overview before more precise adjustments. This helps in identifying major shifts or deformations caused by tumours.

For breast, the initial alignment of the breast mesh sets the stage for accurate mapping of tissue structures.

At the step 336, matching the 3D mesh with the DICOM images may be analyzed. For example, in breast imaging, initial alignment may help in ensuring the accurate representation of tissue layers for density assessment.

Further, upon alignment, the process 300 may include verifying, by the mesh alignment and registration module 122, the 3D mesh model for the close alignment or make some minor adjustments if requirement, to match the DICOM images accurately, at step 338. The step 336 may prepare the 3D mesh model for precise registration which is essential for the accurate representation of the anatomical structures. An Alignment evaluation algorithm may be used to determine if the initial alignment may be sufficient or if it requires any further refinements.

In continuation with the above example, for cardiac, the heart 3D mesh may be evaluated to see if it's close enough for the fine-tuning. This ensures precision in measuring heart valve or artery structures.

For brain, the brain 3D mesh's alignment may be checked to prepare for detailed registration, which is essential for pinpointing tumour(s) or vascular malformations.

For breast, a breast 3D mesh alignment may be assessed to ensure that tumour(s) or dense areas may be accurately positioned.

At the step 338, ensuring the anatomical features are close to the 3D mesh model. By way of an example, in brain imaging, the step may ensure tumours or lesions may be accurately located for surgical planning.

Further, the process 300 may include fine registering, by the mesh alignment and registration module 122, the 3D mesh model, at step 340. Fine registration step may be performed to align the 3D mesh model precisely with the DICOM image slices. The fine registration may also be performed to ensure that the 3D mesh model is as anatomically accurate as possible and supports reliable diagnostics and analysis. Advanced registration algorithms (such as iterative closest point (ICP) and mutual information techniques may be used for the precise alignment.

In continuation with the above example, for cardiac, fine registration may align the heart 3D mesh with the original images for precise measurement of anatomical features like septal defects or valve orientation.

For brain, detailed registration may ensure that the 3D mesh model may accurately represents brain structures, which is crucial for assessing tumour infiltration or brain volume loss.

For breast, the step 340 may refine the alignment of the breast mesh to ensuring that tumour dimensions and locations may be precisely mapped.

At the step 340, final alignment adjustments may be analyzed. By way of an example, in cardiac imaging, fine registration may ensure that coronary arteries may be correctly aligned with the DICOM images to measure blockages or plaque buildup.

Further, once the 3D mesh model is fine registered, the process 300 may include checking, by the mesh alignment and registration module 122, anatomical accuracy of the 3D mesh model, at step 342. The 3D mesh model may be validated against the known anatomical structures (i.e., the clinically verified 3D mesh geometry), at step 342. The step 342 may ensure that the structure features (such as heart valves or brain tumours) may be correctly modelled. This may also ensure that the 3D mesh model's accuracy and make it suitable for clinical use. A validation algorithm may be used to compare the 3D mesh model with standard anatomical models to identify any deviations.

In continuation with the above example, for cardiac, the heart 3D mesh model may be validated against the known anatomical standards. Wall thickness, chamber size, and artery orientation may be checked for accuracy.

For brain, the brain's 3D mesh model may be compared to a standard anatomy to ensure that regions like the hippocampus or brainstem may be correctly represented.

For breast, the breast 3D mesh model may be checked for anatomical accuracy to ensure that suspicious areas may be accurately depicted for biopsy or surgery.

The step 342 may be performed to verify whether the 3D mesh model is anatomically accurate or not. For example, in brain imaging, checking that the ventricles are correctly shaped and positioned for volume measurement.

Further, the process 300 may include validating, by the mesh alignment and registration module 122, the 3D mesh model, at step 344. The 3D mesh model may undergo thorough validation to confirm that all the anatomical features meet the clinical standards, at step 344. Final validation may ensure that the 3D mesh model may be ready for medical analysis or surgical planning. A statistical shape analysis algorithm may evaluate the 3D mesh model against population-based models to ensure clinical accuracy.

In continuation with the example, for cardiac, final validation may ensure that the heart's 3D mesh model is suitable for clinical analysis. Additionally, the final validation may confirm the accuracy of measurements (such as ejection fraction or artery narrowing).

For brain, the brain's 3D mesh model undergoes validation to confirm that tumour size and location are correct, making it reliable for neurosurgical planning.

For breast, the breast's 3D mesh model may be validated for accurate depiction of tissue structures to ensure reliable diagnostics and treatment planning.

At the step 344, overall 3D mesh model accuracy and reliability may be analyzed. By way of an example, in breast imaging, validating that all masses are accurately represented for proper diagnosis.

Further, upon final validation, the process 300 may include checking (or reviewing), by the mesh alignment and registration module 122, the alignment accuracy of the 3D mesh model, at step 346. The final 3D mesh model and the DICOM image slices may be received as an input. The alignment of the 3D mesh model may be reviewed and adjusted for maximum accuracy and make ensure that the 3D mesh model is suitable for further analysis, at step 346. In some embodiments, upon checking, if some misalignment may be detected, then suitable adjustments may be made. The step 346 may ensure the 3D mesh model's alignment may be perfect and support the precise diagnostics. An accuracy assessment algorithm may be used to check for alignment precision and make adjustments as needed.

In continuation with the above example, for cardiac, detailed analysis may ensure the heart mesh is perfectly aligned and may be ready for advanced diagnostic applications like virtual surgery planning.

For brain, the alignment of the brain model may be reviewed to ensure precision, especially important for surgical simulations.

For breast, the breast model's alignment may be checked to ensure that it is accurate for evaluating tumour spread or planning treatment.

The step 346 may ensure that the alignment of the 3D mesh model is precise and ready for the further processing. By way of an example, in cardiac imaging, verifying that all chambers and valves are correctly oriented.

Further, upon validation, the process 300 may include texture mapping for the 3D mesh model, at step 348. The step 348 may include further steps 350, 352, 354, 356, 358, and 360. The process 300 may include projecting, by the texture mapping module 124, textures onto the 3D mesh model, at step 350. The textures may be projected onto the 3D mesh model for adding details to the features (such as myocardial scarring or brain lesions), at step 350. The texture mapping may enhance the 3D mesh model's realism and aid in the visualization and analysis of anatomical structures. A texture mapping algorithm may be used to project 2D textures onto the 3D surface to maintain the anatomical correspondence.

In continuation with the above example, for cardiac, projecting the DICOM textures onto the heart 3D mesh model to add realism, highlighting features (such as myocardial scarring or calcified plaques).

For brain, the texture mapping may highlight brain structures (such as blood vessels and cortical folds) which is useful for vascular studies.

For breast, the textures may enhance the visualization of dense tissue and calcifications, aiding in a more comprehensive assessment of breast cancer.

At the step 350, projecting surface textures on the 3D mesh model for realism may be analyzed. By way of an example, in heart imaging, projecting textures onto the heart mesh to highlight areas of calcification or scarring

Further, upon projecting the textures onto the 3D mesh model, the process 300 may include validating, by the textures mapping module 124, the projected textures, at step 352. The projected textures may be validated to ensure that they may correspond to the anatomical features (such as heart scars, or brain tumours). The step 352 may ensure that the 3D mesh model may be reliable for clinical applications (e.g., planning treatments or surgeries). A correspondence verification algorithm may be used to validate the projected textures onto the 3D mesh model to ensure that the textures may align accurately with the anatomical 3D mesh.

By way of an example, for cardiac, the heart's 3D mesh model is checked for accurate correspondence with textures to ensure that any calcified plaques or scarring may be correctly placed.

For brain, the brain model's (i.e., 3D mesh model) textures may be reviewed to ensure that lesions or abnormalities align with the mesh accurately.

For breast, the textures correspondence may be validated to ensure that all masses or dense areas may be correctly represented.

The step 352 may ensure that the textures may be aligned with the anatomical structures. By way of an example, in brain imaging, ensuring tumours textures match the actual lesion's surface features.

Further, upon validation, the process 300 may include analyzing, by the textures mapping module 124, tissue characteristics, at step 354. The tissue characteristics may be analyzed to differentiate between healthy and diseased tissue using the 3D mesh as a reference model. The step 354 may provide insights into conditions (such as cardiac scarring, brain tumours composition, or breast tissue density). A machine learning model may be used to analyzed tissue properties to classify them as healthy or pathological.

By way of an example, the tissue characteristics may be density (e.g., used to differentiate between soft tissue, bone, and air. In breast imaging, tissue density is crucial for identifying areas of increased cancer risk), elasticity (e.g., assessed through techniques like elastography, where differences in tissue stiffness can indicate tumors or fibrotic tissues. For example, cancerous tissues are often stiffer than surrounding healthy tissue), perfusion (e.g., refers to blood flow within tissues, measured using perfusion imaging. Abnormal patterns can indicate issues like ischemia or tumors), attenuation (e.g., describes how tissue absorbs X-rays in CT scans. For instance, fat tissue has lower attenuation compared to muscle), signal intensity (e.g., in MRI, tissues have different signal intensities. For example, cerebrospinal fluid appears dark on T1-weighted images and bright on T2-weighted images), contrast uptake (e.g., the ability of tissue to absorb contrast agents, with tumors often taking up more contrast), water content (e.g., assessed in MRI; changes can indicate edema or inflammation), fat content (e.g., important for diagnosing conditions like fatty liver disease or distinguishing lipomas from malignant tumors), fibrosis (e.g., detected as thickening and scarring of tissue, seen in ultrasound or MRI), calcification (e.g., calcium deposits detected in tissues, often seen on X-rays and CT scans), tissue composition (e.g., analyzes the ratio of different tissue types, affecting risk assessments), morphology (e.g., the shape and structure of tissues, revealing abnormalities like tumor growth), and the like.

In continuation with the above example, for cardiac, tissue analysis may be configured to distinguish between healthy myocardium and scarred tissue which may be used in the assessment of heart disease.

For brain, texture analysis may be configured to differentiat between tumour tissue and normal brain matter to provide insight into tumour aggressiveness or tissue damage.

For breast, tissue characteristics may be analysed to distinguish between benign and malignant tumour s based on density and texture.

The step 354 may be performed to examine the tissue properties. By way of an example, for heart, scar tissue, perfusion, and myocardial elasticity. The step 354 may identify scarred areas of the myocardium that affect heart function. For brain, tumour consistency, water content, and perfusion. The step 354 may assess whether a tumour is solid or cystic based on MRI signal intensity. For breast, tissue density, mass composition, and vascularity. The step 354 may distinguish between benign and malignant masses based on density and vascular patterns.

Further, once the tissue characteristics are analyzed, the process 300 may include final checking, by the textures mapping module 124, the 3D mesh model to ensure that the 3D mesh model may be accurately represents the anatomical structures, at step 356. The step 356 may confirm that the 3D mesh model may be ready for clinical or research use. Additionally, the step 356 may ensure that all the anatomical structures may be accurately depicted. A final validation algorithm may be used to compare the 3D mesh model with clinical standards to ensure it is accurate and reliable.

In continuation with the above example, for cardiac, the final check may ensure that the heart's 3D mesh model accurately represents all structures including chambers and coronary arteries.

For brain, the brain's 3D mesh model may be validated to ensure all anatomical features are correctly represented for clinical use.

For breast, the breast's 3D mesh model undergoes a final accuracy check to ensure that the breast's 3D mesh model is suitable for diagnostic and therapeutic applications.

At step 356, final validation of the anatomical features may be analyzed. For example, in cardiac imaging, ensuring that the left atrium and ventricles are correctly proportioned.

Further, upon final checking of the 3D mesh model, the process 300 may include mapping, by the textures mapping module 124, the final textures on the 3D mesh model, at step 358. The final textures may be mapped on the 3D mesh model to highlight critical features. This step (i.e., 358) may be performed to complete the 3D mesh model and make them suitable for medical use, research, or education. An optimized texture mapping algorithm may be use to finalize the model's visual representation.

In continuation with the above example, for cardiac, the final textures may be applied to the heart model (i.e., the 3D mesh model) to highlight areas of interest (e.g., valve calcifications or myocardial infarctions).

For brain, the brain model's textures may be finalized to make features like tumour s and vascular structures clear for analysis.

For breast, the breast textures may be applied to provide a realistic representation of tissue structures, aiding in comprehensive diagnostics.

At the step 358, applying and verifying the surface details may be analyzed. By way of an example, in breast imaging, applying textures to highlight dense tissue regions.

Further, upon mapping, the process 300 may include reviewing, by the texture mapping module 124, anatomical representation of the 3D mesh model, at step 360. The 3D mesh model may be reviewed for anatomical accuracy, or confirming the 3D mesh model is ready for use. The step 360 may be performed to ensure the 3D mesh model is reliable for diagnostic, therapeutic, or educational applications. A quality assurance algorithm may be used to perform a final check to validate the 3D mesh model's accuracy and readiness for clinical deployment. In some embodiments, if the 3D mesh model may not be ready for use, then the step 358 may be repeated again till the 3D mesh model become ready for use.

In continuation with the above example, for cardiac, the heart model may be reviewed to ensure that the 3D mesh model may represent the anatomical structures accurately and is ready for clinical or research use.

For brain, the brain model may be checked to confirm its readiness for applications like surgical planning or research.

For breast, the breast model may be validated for anatomical accuracy, ensuring it is suitable for cancer diagnosis and treatment planning.

The step 360, ensuring the final 3D model is anatomically accurate. By way of an example, in brain imaging, confirming the representation of gyri and sulci for accurate neurological assessment.

At last, the process 300 may include rendering the 3D mesh model for one of the visualization, surgical planning, or diagnosis analyzing on a user device (e.g., computer).

Various embodiments provide method and system for constructing a dynamic 3D mesh model of an anatomical region. The disclosed method and system may construct a 3D volume of the anatomical region based on a plurality of DICOM image slices of the anatomical region and a verified 3D mesh model of the anatomical region. Moreover, the disclosed method and system may segment, based on the verified 3D mesh model, the 3D volume to generate one or more segmented volumes using a deep-learning based volume segmentation model. Each of the one or more segmented volumes may include an anatomical structure of the anatomical region. The deep-learning based volume segmentation model is trained, using historical data, to identify the anatomical structure and segment the 3D volume based on the identified anatomical structure. Thereafter, the disclosed method and system may construct a dynamic 3D mesh model of the anatomical region based on the one or more segmented volumes. The dynamic 3D mesh model is indicative of structural and functional characteristics of the anatomical region.

As will be appreciated by those skilled in the art, the techniques described in the various embodiments discussed above are not routine, or conventional, or well understood in the art. The techniques discussed above provide for constructing a dynamic 3D mesh model of an anatomical region. By sharing immersive and interactive 3D models, physicians, radiologists, and surgeons can collaborate seamlessly, discuss cases, and exchange valuable insights. The clinically verified 3D mesh geometry helps ensure that the DICOM slices are aligned correctly and maintain the correct anatomical relationships. This is particularly useful in complex structures like the heart, where chambers and valves need to be accurately positioned. The described techniques assist in segmentation. The mesh provides detailed anatomical information that segmentation algorithms can use to isolate structures more accurately. The described techniques validate and refine spatial relationships. The mesh helps verify that reconstructed 3D models maintain anatomical accuracy, comparing the reconstructed data against a known, verified standard. Additionally, the described techniques enhance the analysis of pixel data by providing a spatially accurate reference that helps identify, segment, and contextualize anatomical features. This integration improves the precision and reliability of the diagnostic process.

In light of the above-mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

It will be appreciated that, for clarity purposes, the above description has described embodiments of the invention with reference to different functional units and processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

Although the present invention has been described in connection with some embodiments, it is not intended to be limited to the specific form set forth herein. Rather, the scope of the present invention is limited only by the claims. Additionally, although a feature may appear to be described in connection with particular embodiments, one skilled in the art would recognize that various features of the described embodiments may be combined in accordance with the invention.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope and spirit of disclosed embodiments being indicated by the following claims.

What is claimed is:

1. A method for constructing a dynamic three-dimensional (3D) mesh model of an anatomical region, the method comprising:
constructing, by an image reconstruction system, a 3D volume of the anatomical region based on a plurality of DICOM image slices of the anatomical region and a verified 3D mesh model of the anatomical region;
segmenting, by the image reconstruction system and based on the verified 3D mesh model, the 3D volume to generate one or more segmented volumes using a deep-learning based volume segmentation model,
wherein each of the one or more segmented volumes comprises an anatomical structure of the anatomical region, and
wherein the deep-learning based volume segmentation model is trained, using historical data, to identify the anatomical structure and segment the 3D volume based on the identified anatomical structure; and
constructing, by the image reconstruction system, a dynamic 3D mesh model of the anatomical region based on the one or more segmented volumes, wherein the dynamic 3D mesh model is indicative of structural and functional characteristics of the anatomical region.

2. The method of claim 1, comprising:
extracting metadata from each of the plurality of DICOM image slices; and
comparing the metadata with corresponding reference data from the verified 3D mesh model, wherein the metadata is indicative of at least one of the structural and the functional characteristics and comprises at least one of a patient orientation, a slice thickness, a slice spacing, one or more imaging parameters, one or more spatial coordinates, a spatial alignment, a field of view, an image resolution.

3. The method of claim 2, comprising adjusting the plurality of DICOM image slices based on the comparison, wherein adjusting comprises at least one of: reordering a sequence of one or more of the plurality of DICOM image slices, correcting a spatial alignment of one or more of the plurality of DICOM image slices, correcting a spatial orientation of one or more of the plurality of DICOM image slices, adjusting a slice thickness of one or more of the plurality of DICOM image slices, interpolating one or more missing slices based on surrounding slices from the plurality of DICOM image slices, or refining one or more corrupted slices based on surrounding slices from the plurality of DICOM image slices.

4. The method of claim 3, wherein adjusting the plurality of DICOM image slices comprises:
extracting pixel data from each of the plurality of DICOM image slices; and
processing the pixel data across the plurality of DICOM image slices based on the verified 3D mesh model to normalize intensity levels of the pixel data.

5. The method of claim 1, wherein constructing the 3D volume comprises:
sorting and aligning the plurality of DICOM image slices based on the verified 3D mesh model to maintain a continuity of an anatomical structure and a spatial relationship among the plurality of DICOM image slices; and
stacking the plurality of DICOM image slices upon sorting and aligning to construct the 3D volume.

6. The method of claim 5, wherein constructing the 3D volume comprises:
validating an alignment of the 3D volume based on metadata extracted from each of the plurality of DICOM image slices.

7. The method of claim 1, wherein segmenting the 3D volume comprises:
validating a quality and a consistency of each of the one or more segmented volumes for an artifact or for a variation in the anatomical structure; and
processing each of the one or more segmented volumes to remove the artifact and smoothen the variation.

8. The method of claim 1, wherein constructing the dynamic 3D mesh model of the anatomical region comprises:
constructing an initial 3D mesh model of the anatomical region based on the one or more segmented volumes;
iteratively aligning the initial 3D mesh model with the plurality of DICOM image slices using spatial registration to generate an aligned 3D mesh model;
validating the aligned 3D mesh model based on the verified 3D mesh model to generate a validated 3D mesh model; and
adjusting an alignment of the validated 3D mesh model based on the plurality of DICOM image slices to generate the dynamic 3D mesh model.

9. The method of claim 1, comprising:
rendering the dynamic 3D mesh model for at least one of a visualization, a diagnostic analysis, or a surgical planning.

10. The method of claim 9, wherein rendering the dynamic 3D mesh model comprises:
mapping textures derived from the plurality of DICOM image slices onto the dynamic 3D mesh model; and
analyzing the textures and the dynamic 3D mesh model to determine tissue characteristics using a machine learning model.

11. A system for constructing a dynamic 3D mesh model of an anatomical region, the system comprising:
a processor; and
a memory communicatively coupled to the processor, wherein the memory stores processor executable instructions, which, on execution, causes the processor to:
construct a 3D volume of the anatomical region based on a plurality of DICOM image slices of the anatomical region and a verified 3D mesh model of the anatomical region;
segment, based on the verified 3D mesh model, the 3D volume to generate one or more segmented volumes using a deep-learning based volume segmentation model,
wherein each of the one or more segmented volumes comprises an anatomical structure of the anatomical region, and
wherein the deep-learning based volume segmentation model is trained, using historical data, to identify the anatomical structure and segment the 3D volume based on the identified anatomical structure; and
construct a dynamic 3D mesh model of the anatomical region based on the one or more segmented volumes, wherein the dynamic 3D mesh model is indicative of structural and functional characteristics of the anatomical region.

12. The system of claim 11, wherein the processor executable instructions further cause the processor to:
extract metadata from each of the plurality of DICOM image slices; and
compare the metadata with corresponding reference data from the verified 3D mesh model, wherein the metadata is indicative of at least one of the structural and the functional characteristics and comprises at least one of a patient orientation, a slice thickness, a slice spacing, one or more imaging parameters, one or more spatial coordinates, a spatial alignment, a field of view, an image resolution.

13. The system of claim 12, wherein the processor executable instructions further cause the processor to adjust the plurality of DICOM image slices based on the comparison, wherein to adjust, the processor executable instructions cause the processor to, at least one of:
reorder a sequence of one or more of the plurality of DICOM image slices,
correct a spatial alignment of one or more of the plurality of DICOM image slices, correct a spatial orientation of one or more of the plurality of DICOM image slices,
adjust a slice thickness of one or more of the plurality of DICOM image slices,
interpolate one or more missing slices based on surrounding slices from the plurality of DICOM image slices, or
refine one or more corrupted slices based on surrounding slices from the plurality of DICOM image slices.

14. The system of claim 13, wherein to adjust the plurality of DICOM image slices, the processor executable instructions cause the processor to:
extract pixel data from each of the plurality of DICOM image slices; and
process the pixel data across the plurality of DICOM image slices based on the verified 3D mesh model to normalize intensity levels of the pixel data.

15. The system of claim 11, wherein to construct the 3D volume, the processor executable instructions cause the processor to:
sort and align the plurality of DICOM image slices based on the verified 3D mesh model to maintain a continuity of an anatomical structure and a spatial relationship among the plurality of DICOM image slices; and
stack the plurality of DICOM image slices upon sorting and aligning to construct the 3D volume.

16. The system of claim 15, wherein to construct the 3D volume, the processor executable instructions cause the processor to:
validate an alignment of the 3D volume based on metadata extracted from each of the plurality of DICOM image slices.

17. The system of claim 11, wherein to segment the 3D volume, the processor executable instructions cause the processor to:
validate a quality and a consistency of each of the one or more segmented volumes for an artifact or for a variation in the anatomical structure; and
process each of the one or more segmented volumes to remove the artifact and smoothen the variation.

18. The system of claim 11, wherein to construct the dynamic 3D mesh model of the anatomical region, the processor executable instructions cause the processor to:
construct an initial 3D mesh model of the anatomical region based on the one or more segmented volumes;
iteratively align the initial 3D mesh model with the plurality of DICOM image slices using spatial registration to generate an aligned 3D mesh model;
validate the aligned 3D mesh model based on the verified 3D mesh model to generate a validated 3D mesh model; and
adjust an alignment of the validated 3D mesh model based on the plurality of DICOM image slices to generate the dynamic 3D mesh model.

19. The system of claim 11, wherein the processor executable instructions further cause the processor to:
render the dynamic 3D mesh model for at least one of a visualization, a diagnostic analysis, or a surgical planning.

20. The system of claim 19, wherein to render the dynamic 3D mesh model, the processor executable instructions cause the processor to:
map textures derived from the plurality of DICOM image slices onto the dynamic 3D mesh model; and
analyze the textures and the dynamic 3D mesh model to determine tissue characteristics using a machine learning model.
